(19) United States
(12) Patent Application Publication
(10) Pub. No.: US 2026/0014074 A1
(43) Pub. Date: Jan. 15, 2026

(54) A METHOD FOR ENHANCING SOLUBILITY OF TRANEXAMIC ACID

(71) Applicant: RAFA LABORATORIES LTD., Jerusalem (IL)

(72) Inventors: Tzufit WARAFTIG, Mevo Horon (IL); Ella ZURIEL, Jerusalem (IL)

(21) Appl. No.: 18/998,760

(22) PCT Filed: Jul. 13, 2023
(86) PCT No.: PCT/IL2023/050739

§ 371 Date: Jan. 27, 2025

(30) Foreign Application Priority Data
Jul. 27, 2022 (IT) 102022000015858

(51) Int. Cl. A61K 9/08; A61K 31/195; A61K 47/02

(52) U.S. Cl. CPC A61K 9/08; A61K 31/195; A61K 47/02

(57) ABSTRACT

A method for enhancing solubility of tranexamic acid comprising the following steps:
a) Initially dissolving tranexamic acid in water;
b) Measuring a pH value of the solution obtained in Step (a);
c) Addition of a strong acid (that dissociates completely in water) to the solution obtained in Step (a) and simultaneously monitoring the decrease in the pH value of the solution until approximately pH 4.5 or less than pH 4.5 is reached, to increase solubility of tranexamic acid;
d) Addition of tranexamic acid to reach the pH value of the solution obtained in Step (c) above the pH 2.0; and
e) Repeating Steps (c) and (d) until a desired amount of tranexamic acid is added to the solution of Step (a), that is a desired concentration of tranexamic acid in the final solution is reached.

### TECHNICAL FIELD

In general, the present application relates to the field of therapeutic dosage forms and preparing the same. In particular, the present application relates to a method of enhancing solubility of tranexamic acid and preparing its liquid dosage forms.

### BACKGROUND

Tranexamic acid is a medicine that controls bleeding and used to treat or prevent excessive blood loss from major trauma, postpartum bleeding, surgery and tooth removal. It helps blood to clot and is used for nosebleeds and heavy periods. For example, once having a tooth taken out, using tranexamic acid mouthwash can help stop bleeding. Tranexamic acid can also help resolve the condition that causes swelling under the skin (hereditary angioedema). In case of any condition that causes bleeding more than usual, tranexamic acid may be prescribed for a few days before having surgery or dental surgery.

Tranexamic acid normally comes on prescription as tablets, liquid dosage forms for children and people who cannot swallow tablets, mouthwash and injections usually only given in hospitals.

Tranexamic acid is marketed in the U.S. and Australia in a tablet form as Lysteda and in Australia and Sweden, it is marketed in an IV form and tablet form as Cyklokapron, in the UK and Sweden as Cyclo-F. In the UK it is also marketed as Femstrual, in Asia as Transcam, in India as Pause, in South America as Espercil, in Japan as Nicolda, in France, Poland, Belgium and Romania as Exacyl. In the Philippines, its capsule form is marketed as Hemostan and in Israel as Hexakapron.

The tablet dosage forms of tranexamic acid come as 500-mg tablets. For adults the usual dose is 2 or 3 tablets, taken 3 times a day. Doses are usually lower for people with kidney problems. The liquid dosage forms come in different strengths. For example, Cyklokapron is administered as an injectable solution of 100 mg/mL. However, frequently, due to enhanced bleeding or complication connected to surgery or other aforementioned conditions, there is an urgent need for higher concentrated dosages of tranexamic acid. Unfortunately, such dosages are not available today due to relatively low solubility of tranexamic acid in water, which is about 167 mg/mL at standard conditions. Therefore, there is long-felt need to develop liquid dosages of tranexamic acid having concentrations higher than its solubility.

### SUMMARY

The present invention describes embodiments of a method for enhancing solubility of tranexamic comprising the following steps:
a) Initially dissolving tranexamic acid in water;
b) Measuring a pH value of the solution obtained in Step (a);
c) Addition of a strong acid (that dissociates completely in water) to the solution obtained in Step (a) and simultaneously monitoring the decrease in the pH value of the solution until approximately pH 4.5 or less than pH 4.5 is reached, to increase solubility of tranexamic acid;
d) Addition of tranexamic acid to reach the pH value of the solution obtained in Step (c) above the pH 2.0; and
e) Repeating Steps (c) and (d) until a desired amount of tranexamic acid is added and dissolved in the solution of Step (a), that is a desired concentration of tranexamic acid in the final solution is reached.

In some embodiments, the initial concentration of tranexamic acid dissolved in water in Step (a) of the method of the present invention is about 120-200 mg/mL. In a particular embodiment, the initial concentration of tranexamic is about 130-180 mg/mL, or 130-170 mg/mL, or 130-160 mg/mL. In a specific embodiment, the initial concentration of tranexamic acid is about 130 mg/mL.

In other embodiments, the pH value of the solution of Step (a) is lowered in Step (c) to approximately pH 4.5 or below pH 4.5. In certain embodiments, the pH value of the solution of Step (a) is lowered in Step (c) below pH 4.0, or below, 3.5, or below pH 3.0, or below pH 2.5, or below pH 2.0, or below pH 1.5, or below pH 1.0, or below pH 0.5.

In further embodiments, the strong acid used in the present invention to decrease in the pH value of the solution in Step (c), thereby increasing solubility of tranexamic acid, is selected from hydrochloric acid, perchloric acid, sulfuric acid, nitric acid, hydrobromic acid, hydroiodic acid, chloric acid, and phosphoric acid. The concentration of said strong acid is about 0.01 to 10.00 eq/L.

In yet further embodiments, the pH value of the solution of Step (c) raises in Step (d) with addition of more tranexamic acid above pH 2.5, or above pH 3.0, or above pH 3.5, or above pH 4.0, or above pH 4.5, or above pH 5.0, or above pH 5.5. In a specific embodiment, the pH raises in Step (d) to approximately pH 3.5.

In still other embodiments, the desired concentration of tranexamic acid in the final solution is above 160 mg/mL, or above 250 mg/mL, or above 350 mg/mL, or above 400 mg/mL, or in the range about 200-600 mg/mL, or in the range about 300-500 mg/mL. In a specific embodiment, the desired concentration of tranexamic acid in the final solution is approximately 400 mg/mL, or specifically 440 mg/mL.

In another aspect, the present invention relates to a liquid dosage form comprising tranexamic acid in a concentration higher than 160 mg/mL, or higher than 250 mg/mL, or higher than 350 mg/mL, or higher than 400 mg/mL, or in the range about 200-600 mg/mL, or in the range about 300-500 mg/mL. In a specific embodiment, the liquid dosage form of the present invention comprises tranexamic acid in a concentration of approximately 400 mg/mL, or specifically 440 mg/mL.

Various embodiments may allow various benefits and may be used in conjunction with various applications. The details of one or more embodiments are set forth in the accompanying description below. Other features, objects and advantages of the described techniques will be apparent from the description and from the claims.

### DETAILED DESCRIPTION

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a method comprising the steps x and z” should not be limited to methods consisting only of these steps.

Unless specifically stated, as used herein, the terms “about” and “approximately” are understood as within a range of normal tolerance in the art, for example within two standard deviations of the mean. In one embodiment, the terms “about” and “approximately” mean within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The present invention relates to a method for enhancing solubility of tranexamic comprising the following steps:
a) Initially dissolving tranexamic acid in water;
b) Measuring a pH value of the solution obtained in Step (a);
c) Addition of a strong acid (that dissociates completely in water) in drops to the solution obtained in Step (a) and simultaneously monitoring the decrease in the pH value of the solution until approximately pH 4.5 or less than pH 4.5 is reached, to increase solubility of tranexamic acid;
d) Addition of tranexamic acid to raise the pH value of the solution obtained in Step (c) above the pH 2.0; and
e) Repeating Steps (c) and (d) until a desired amount of tranexamic acid is added and dissolved in the solution of Step (a), that is a desired concentration of tranexamic acid in the final solution is reached.

Tranexamic acid is an organic acid having the following formula:

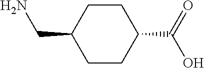 [SMILES: NC[C@H]1CC[C@H](C(=O)O)CC1]

Its IUPAC name: trans-4-(aminomethyl)cyclohexanecarboxylic acid, and CAS No. 1197-18-8.

Tranexamic acid is a synthetic analogue of the amino acid lysine. It serves as an antifibrinolytic by reversibly binding four to five lysine receptor sites on plasminogen. This decreases the conversion of plasminogen to plasmin, preventing fibrin degradation and preserving the framework of fibrin's matrix structure. Therefore, it is used as a medicine for blood clotting, controls bleeding and used to treat or prevent excessive blood loss from major trauma, postpartum bleeding, surgery and tooth removal.

It is important to note that solubility of tranexamic acid is a limiting factor in all dosage forms which are commercially available nowadays. Therefore, the liquid dosage forms of tranexamic acid having higher concentration than approximately 180 mg/mL are not known. It was unexpected discovery made by the present inventors that the aforesaid method of the present invention allows to significantly increase solubility of tranexamic acid in its liquid dosage forms, which surprisingly appeared to be very stable and having a long shelf-life.

In some embodiments, the initial concentration of tranexamic acid dissolved in water in Step (a) of the method of the present invention is about 120-200 mg/mL. In a particular embodiment, the initial concentration of tranexamic is about 130-180 mg/mL, or 130-170 mg/mL, or 130-160 mg/mL. In a specific embodiment, the initial concentration of tranexamic acid is about 130 mg/mL.

In other embodiments, the pH value of the solution of Step (a) is lowered in Step (c) to approximately pH 4.5 or below pH 4.5. In certain embodiments, the pH value of the solution of Step (a) is lowered in Step (c) below pH 4.0, or below pH 3.5, or below pH 3.0, or below pH 2.5, or below pH 2.0, or below pH 1.5, or below pH 1.0, or below pH 0.5. In a specific embodiment, the pH value of the solution of Step (a) is lowered in Step (c) to approximately pH 0.2.

In further embodiments, the strong acid used in the present invention to decrease in the pH value of the solution in Step (c), thereby increasing solubility of tranexamic acid, is selected from hydrochloric acid, perchloric acid, sulfuric acid, nitric acid, hydrobromic acid, hydroiodic acid, chloric acid, and phosphoric acid. The concentration of said strong acid is about 0.01 to 10.00 eq/L.

In yet further embodiments, the pH value of the solution of Step (c) raises in Step (d) with, and as a result of, addition of more tranexamic acid above pH 2.5, or above pH 3.0, or above pH 3.5, or above pH 4.0, or above pH 4.5, or above pH 5.0, or above pH 5.5. In a specific embodiment, the pH raises in Step (d) to approximately pH 3.5. In a particular embodiment, the pH is raised in Step (d) to approximately pH 4.5.

In still other embodiments, the desired concentration of tranexamic acid in the final solution is above 160 mg/mL, or above 250 mg/mL, or above 350 mg/mL, or above 400 mg/mL, or in the range about 200-600 mg/mL, or in the range about 300-500 mg/mL. In a specific embodiment, the desired concentration of tranexamic acid in the final solution is approximately 400 mg/mL, or specifically 440 mg/mL.

In another aspect, the present invention relates to a liquid dosage form comprising tranexamic acid in a concentration higher than 160 mg/mL, or higher than 250 mg/mL, or higher than 350 mg/mL, or higher than 400 mg/mL, or in the range about 200-600 mg/mL, or in the range about 300-500 mg/mL. In a specific embodiment, the liquid dosage form of the present invention comprises tranexamic acid in a concentration of approximately 400 mg/mL, or specifically 440 mg/mL.

In still another aspect, the present invention relates to a liquid dosage form preparing by the method of the present invention and comprising tranexamic acid in a concentration higher than 160 mg/mL, or higher than 250 mg/mL, or higher than 350 mg/mL, or higher than 400 mg/mL, or in the range about 200-600 mg/mL, or in the range about 300-500 mg/mL. In a specific embodiment, the liquid dosage form of the present invention comprises tranexamic acid in a concentration of approximately 400 mg/mL, or specifically 440 mg/mL.

In an additional embodiment of the present invention, the method of the invention comprises the following steps:
a) Dissolving tranexamic acid in water with the concentration of about 120-200 mg/mL;
b) Measuring a pH value of the solution obtained in Step (a);
c) Addition of a strong acid selected from hydrochloric acid, perchloric acid, sulfuric acid, nitric acid, hydrobromic acid, hydroiodic acid, chloric acid, and phosphoric acid, in drops to the solution obtained in Step (a) and simultaneously monitoring the decrease in the pH value of the solution until approximately pH from 3.0 to 4.0 is reached, to increase solubility of tranexamic acid;
d) Addition of tranexamic acid to raise the pH value of the solution obtained in Step (c) to approximately pH from 3.5 to 4.5; and
e) Repeating Steps (c) and (d) until the concentration of tranexamic acid in the final solution reaches approximately 250-440 mg/mL.

What is unique about the method of the present invention is not only that it allows to prepare aqueous solutions of tranexamic acid dissolved at a higher concentration than its solubility in water. These highly concentrated solutions of TXA were found to be very stable at the following conditions: 3 months at 40° C., as demonstrated in the following experimental examples.

### Example 1

Stability Data—Tranexamic Acid (TXA) 400 mg/ml, 2.5 ml Injector
Batch no. D-2363
Dosage: 400 mg/ml, 2.5 ml
Conditions: 40° C./75% RH

The examination of the stability of the drug product was evaluated by comparing the stability data to current BP monograph for TXA injections and to assured that the results within the acceptance criteria throughout the product shelf life. It should be noted that the product is clear which indicate that the solution is pure, and all the material are dissolved completely.

The stability data are presented in the following table:

TABLE 1
Stability: 40° C./75% RH 3 M

| | Specifica- | Time | | | |
|---|---|---|---|---|---|
| Test | tions¹ | zero | 1 M | 3 M | 6 M |
| Appearance | Colorless to | Pass | Pass | Pass | Pass |
| | slight yellow | | | | |
| | clear | | | | |
| | solution | | | | |
| Assay | 95.0-105.0% | 98.1% | 97.1% | 99.1% | 98.9% |
| Impurity A | NMT 0.1% | <0.05% | <0.05% | <0.05% | <0.05% |
| Impurity B | NMT 0.1% | 0.012% | 0.097% | 0.089% | 0.08% |
| (cis- | | | | | |
| Tranexamic | | | | | |
| acid) | | | | | |
| Impurity C | NMT 0.1% | <0.05% | <0.05% | <0.05% | <0.05% |
| Impurity D | NMT 0.1% | <0.05% | <0.05% | <0.05% | <0.05% |
| (aminoethyl | | | | | |
| benzoic acid) | | | | | |
| Impurity E | NMT 0.15% | 0.06% | <0.05% | <0.05% | <0.05% |
| Impurities | NMT 2.0% | 0.12% | 0.1% | <0.05% | <0.05% |
| total | | | | | |
| pH | TBD | 3.6 | 3.7 | 3.3 | 3.5 |

¹Wherever possible, specification limits where set according to BP monograph for TXA injection.

For IMP B— According to ICHQ3B(R2) it was set to 0.2% instead of 0.5% due to the MDD (maximum daily dose). Impurity E—is not part of the BP monograph so according to ICHQ3B(R2) it was set to 0.15%.
   1. Appearance—colourless to slightly yellow clear solution.

No change has been seen in the appearance of the product.

2. Assay—between 95.0% and 105.0% of the declared concentration for TXA.

All assay results stay well within the specified limits. It is a common approach for such products to define those limits for assay.

3. pH

The pH throughout the 6M stability is stable and did not vary significantly.

The limit of the pH will be evaluated the during the development studies.

4. Impurities and Degradation products:

4.1 Impurity “B” cis-Tranexamic acid—NMT 0.1%

Impurity “B” is an isomer of TXA. The results are within the limits after 6-months.

4.2 Impurity “C”—NMT 0.1%.

The results are within limits.

4.3 Impurity “D” aminomethyl benzoic acid—NMT 0.1%.

The results are within limits.

4.4 Impurity total—NMT 2.0%.

The results are within limits.

### Conclusion

The product TXA 400 mg/ml, in 2.5 ml injector is a stable product after 6 months in accelerate conditions (40° C./75% RH) with no significant increase in the degradation products and no significant decrease in the assay levels.

### Example 2

Release Data—TXA 400 mg/ml, 2.5 ml
Batch no. D-2565
Dosage: 400 mg/m, 2.5 ml
Trial description: pH value throughout the process was approximately pH 4

The purpose of the trial was to ensure that ability of the TXA to dissolve is possible also at pH 4.

The release data are presented below:

### Release Date for Batch D-2565

| Test | Specifications | D-2565 |
|---|---|---|
| Appearance | Colorless to slight yellow | Pass |
| | Clear solution | |
| Assay | 95.0-105.0% | 98.4% |
| Impurity A | NMT 0.1% | <0.05% |
| Impurity B (cis-Tranexamic acid) | NMT 0.1% | <0.05% |
| Impurity C | NMT 0.1% | <0.05% |
| Impurity D (Aminoethyl benz. Acid) | NMT 0.1% | <0.05% |
| Impurity E | NMT 0.15% | ND |
| Impurity unknown | NMT 0.1% | ND |
| Impurities total | NMT 2.0% | <0.05% |
| PH | TBD | 4.1 |

### Conclusion

The product TXA 400 mg/ml, in 2.5 ml injector can be produce with acidity of approximately pH 4. In addition to the clear solution that indicate full solubility of the TXA, the analytical results met the acceptance criteria.

### Example 3

### Preparing the Formulation of Tranexamic Acid by the Method of the Invention

1. Dissolving the 135 mg/ml in water
2. Decreasing the pH value of the solution to approximately 3.0 with strong acid (HCl 5N)
3. Adding tranexamic acid until pH reached approximately 4.0.
4. Adding strong acid (HCl 5N) to reduce pH to approximately 3.
5. Keep preforming step 3+4 until reaching tranexamic acid concentration of 400 mg/ml.

### Example 4

Leading Process Stability Data—TXA 400 mg/ml, 2.5 ml
Batch no. D-2556
Dosage: 400 mg/ml, 2.5 ml
Conditions: 2 weeks/65oC
Batch D-2556 manufacturer with the intended formulation and process:
1. Weighting WFI
2. Adding TXA and mixing until dissolved
3. Adding HCl to approximately pH 3
4. Adding TXA and mixing until dissolved
5. Repeating stages 3+4 to the desired amount of TXA
6. Weight completion with WFI

The examination of the stability of the drug product was evaluated by comparing the stability data to current BP monograph for TXA injections and to assured that the lead formulation and process are within the acceptance criteria throughout the product shelf life. It should be noted that the product was clear that indicates that the solution is pure, and all the material is dissolved completely.

The batch was stored in accelerated conditions at 65° C. and 40° C.

The stability data of the 2 weeks 65° C. are presented in the table below:

| | | Time | 2 |
|---|---|---|---|
| Test | Specifications¹ | zero | weeks |
| Appearance | Colorless to slight | Pass | Pass |
| | yellow Clear solution | | |
| Assay | 95.0-105.0% | 99.1% | 98.6% |
| Impurity A | NMT 0.1% | <0.05% | <0.05% |
| Impurity B (cis-Tranexamic | NMT 0.1% | <0.05% | <0.05% |
| acid) | | | |
| Impurity C | NMT 0.1% | <0.05% | <0.05% |
| Impurity D (Aminoethyl | NMT 0.1% | <0.05% | <0.05% |
| benz. Acid) | | | |
| Impurity E | NMT 0.15% | ND | 0.074% |
| Impurity unknown | NMT 0.1% | ND | ND |
| Impurities total | NMT 2.0% | <0.05% | 0.07% |
| PH | TBD | 3.6 | 3.7 |

¹Wherever possible, specification limits where set according to BP monograph for TXA injection. For IMP B- According to ICHQ3B(R2) it was set to 0.2% instead of 0.5% due to the MDD (maximum daily dose). Impurity E-is not part of the BP monograph so according to ICHQ3B(R2) it was set to 0.15%.

### 1. Appearance—Colourless to Slightly Yellow Clear Solution.

No change has been seen in the appearance of the product.

### 2. Assay—Between 95.0% and 105.0% of the Declared Concentration for TXA.

All assay results stay well within the specified limits after 2 weeks 65° C.

3. pH

### The pH Throughout the 2 Weeks 65° C. Stability is Stable and Did not Vary Significantly.

### 4. Impurities and Degradation Products:

4.1 Impurity “B” cis-Tranexamic acid—NMT 0.1%

Impurity “B” is an isomer of TXA. The results are within the acceptance criteria and <QL (0.05%) after 2 weeks 65° C.

4.2 Impurity “C”—NMT 0.1%.

The results are within the acceptance criteria and <QL (0.05%) after 2 weeks 65° C.

4.3 Impurity “D” aminomethyl benzoic acid—NMT 0.1%.

The results are within the acceptance criteria and <QL (0.05%) after 2 weeks 65° C.

4.4 Impurity “E”—NMT 0.15%.

The results are within the acceptance criteria after 2 weeks 65° C.

4.5 Impurity unknown—NMT 0.1%.

The results are within the acceptance criteria and <QL (0.05%) after 2 weeks 65° C.

4.6 Impurity total—NMT 2.0%.

The results are within the acceptance criteria after 2 weeks 65° C.

### Conclusion

The product TXA 400 mg/ml, in 2.5 ml is a stable product in accelerate radical conditions (after 2 weeks at 65° C.) with no significant increase in the degradation products and no significant decrease in the assay levels.

1. A method for enhancing solubility of tranexamic acid comprising the following steps:
a) Initially dissolving tranexamic acid in water;
b) Measuring a pH value of the solution obtained in Step (a);
c) Addition of a strong acid (that dissociates completely in water) to the solution obtained in Step (a) and simultaneously monitoring the decrease in the pH value of the solution until approximately pH 4.5 or less than pH 4.5 is reached, to increase solubility of tranexamic acid;
d) Addition of tranexamic acid to reach the pH value of the solution obtained in Step (c) above the pH 2.0; and
e) Repeating Steps (c) and (d) until a desired amount of tranexamic acid is added to the solution of Step (a), that is a desired concentration of tranexamic acid in the final solution is reached.

2. The method of claim 1, wherein the initial concentration of tranexamic acid dissolved in water in Step (a) of the method is about 120-200 mg/mL.

3. The method of claim 2, wherein the initial concentration of tranexamic is about 130-180 mg/mL.

4. The method of claim 3, wherein the initial concentration of tranexamic is about 150-170 mg/mL.

5. The method of claim 4, wherein the initial concentration of tranexamic is about 165 mg/mL.

6. The method of claim 1, wherein the pH value of the solution of Step (a) is lowered in Step (c) to approximately pH 4.0.

7. The method of claim 1, wherein the pH value of the solution of Step (a) is lowered in Step (c) below pH 4.0.

8. The method of claim 7, wherein the pH value of the solution of Step (a) is lowered in Step (c) below pH 3.5, or below pH 3.0, or below pH 2.5, or below pH 2.0, or below pH 1.5, or below pH 1.0, or below pH 0.5.

9. The method of claim 7, wherein the pH value of the solution of Step (a) is lowered in Step (c) to approximately pH 0.2.

10. The method of claim 1, wherein said strong acid is selected from hydrochloric acid, perchloric acid, sulfuric acid, nitric acid, hydrobromic acid, hydroiodic acid, chloric acid, and phosphoric acid.

11. The method of claim 1, wherein the concentration of said strong acid is about 0.01 to 10.00 eq/L.

12. The method of claim 1, wherein the pH value of the solution of Step (c) raises in Step (d) with addition of tranexamic acid above pH 2.5, or above pH 3.0, or above pH 3.5, or above pH 4.0, or above pH 4.5, or above pH 5.0, or above pH 5.5.

13. The method of claim 12, wherein the pH value of the solution of Step (c) raises in Step (d) to approximately pH 3.5.

14. The method of claim 12, wherein the pH value of the solution of Step (c) raises in Step (d) above pH 4.5.

15. The method of claim 1, wherein the desired concentration of tranexamic acid in the final solution is above 160 mg/mL, or above 250 mg/mL, or above 350 mg/mL.

16. The method of claim 15, wherein the desired concentration of tranexamic acid in the final solution is above 400 mg/mL.

17. The method of claim 1, wherein the desired concentration of tranexamic acid in the final solution is about 200-600 mg/mL, or 300-500 mg/mL.

18. The method of claim 17, wherein the desired concentration of tranexamic acid in the final solution is approximately 400 mg/mL.

19. The method of claim 18, wherein the desired concentration of tranexamic acid in the final solution is approximately 450 mg/mL.

20. The method of claim 1, comprising the following steps:
a) Dissolving tranexamic acid in water with the concentration of about 120-200 mg/mL;
b) Measuring a pH value of the solution obtained in Step (a);
c) Addition of a strong acid selected from hydrochloric acid, perchloric acid, sulfuric acid, nitric acid, hydrobromic acid, hydroiodic acid, chloric acid, and phosphoric acid, in drops to the solution obtained in Step (a) and simultaneously monitoring the decrease in the pH value of the solution until approximately pH from 3.0 to 4.0 is reached, to increase solubility of tranexamic acid;
d) Addition of tranexamic acid to raise the pH value of the solution obtained in Step (c) to approximately pH from 3.5 to 4.5; and
e) Repeating Steps (c) and (d) until the concentration of tranexamic acid in the final solution reaches approximately 250-450 mg/mL.
